(19) United States
(12) Patent Application Publication
(10) Pub. No.: US 2022/0017956 A1
(43) Pub. Date: Jan. 20, 2022

(54) ENDOGENOUS COMPLEXITY CALIBRATION LADDER TARGET

(71) Applicant: Accugenomics, Inc., Wilmington, NC (US)

(72) Inventor: Tom B. Morrison, Wilmington, NC (US)

(21) Appl. No.: 17/374,455

(22) Filed: Jul. 13, 2021

Related U.S. Application Data
(60) Provisional application No. 63/051,807, filed on Jul. 14, 2020.

(51) Int. Cl. C12Q 1/6869

(52) U.S. Cl. CPC C12Q 1/6869

(57) ABSTRACT

The present disclosure relates to the technical fields of genetic sequencing, and particularly to a method for determining the level of complexity of next-generation sequencing (NGS) libraries.

### CROSS-REFERENCE TO RELATED APPLICATIONS

This application claims priority to and the benefit of U.S. Provisional Patent Application No. 63/051,807 filed Jul. 14, 2019, the disclosure of which is herein incorporated by reference in its entirety.

### FIELD OF INVENTION

The present disclosure relates to the technical fields of genetic sequencing, and particularly to a method for determining the level of complexity of next-generation sequencing (NGS) libraries.

### BACKGROUND OF THE INVENTION

Next-generation sequencing (NGS) technologies provide insights into sequence and structural variations by achieving unparalleled levels of genome and transcriptome coverage. Library preparation for different NGS applications generally follow the same workflow. The general principle is that more starting material means less amplification and thus better library complexity. In all cases, the goal is to make the libraries as complex as possible.

Rapid advances in library preparation, sequencing chemistry and experimental settings diversify the complexity and quality of sequencing data, but it is difficult, if not impossible, to estimate how well the original sample complexity is captured by the library. The complexity yield provides a direct measure of library capture efficiency, which has important quality control implications when attempting to detect rare events.

For example, a problem with preparing sequencing libraries by PCR amplification is that PCR introduces GC bias, a major source of unwanted variation and errors in the sequencing coverage. Providing unique molecular indexes is good at indicating unique input templates, but does not estimate how well the original sample complexity was captured in the library.

Controlling for technical variation in library preparations allows for a lower limit of detection for variant allele fractions. As such, there is an urgent need for methods to quantify and qualify the level of complexity in NGS libraries that are clinically deployable and have increased analytic sensitivity, simplified workflow, and improved quality control measures.

### SUMMARY OF THE INVENTION

With respect to the technical defect in the prior art, the present disclosure provides a method for determining the complexity yield of a prepared next generation sequencing library.

The technical solutions of the present disclosure will be illustrated in detail hereinafter.

A method of determining the complexity yield of a prepared next generation sequencing (NGS) library, comprising obtaining an amount of sample DNA (sDNA) comprising one or more endogenous target genes; preparing a set of synthetic internal standards for at least one of said target genes, wherein the internal standards are the sequence of the target gene within which a substitution of a number (n) of adjacent bases with all 4 nucleic acid bases (N) is made; comingling a known copy number of the internal standards with the sDNA sample to create a combined sample; preparing a NGS library from the combined sample for sequencing; sequencing the combined sample; and analyzing the sequencing data to measure the number of unique reads corresponding to the internal standards (unique IS), and calculating a complexity yield between the unique IS and the known copy number of internal standards in the combined sample.

In certain embodiments, the method comprises preparing a set of synthetic internal standards for at least one of said target genes, wherein the internal standards are the sequence of the target gene within which a substitution of a number (n) of non-adjacent bases with all 4 nucleic acid bases (N) is made.

According to the above method, the complexity yield indicates the quality of the prepared NGS library.

According to the above method, a reduce of the complexity yield from a previously established normal complicity yield indicates a poorer quality of the prepared NGS library.

According to the above method, a number of sDNA target templates in the NGS library prepared from the combined sample is calculated by multiplying the ratio of unique IS to a total number of control reads (IS depth) with the total number of native reads (NT depth).

According to the above method, the number of sDNA templates in the NGS library is used to calibrate the amount of sDNA needed in the preparation of a second NGS library to provide superior depth of analysis for the one or more endogenous target genes in the sDNA than in the original NGS library.

According to the above method, the amount of sDNA in the second NGS library is adjusted to provide an adequate number of unique reads of one or more endogenous target genes to provide variant allele frequency sensitivity.

According to the above method, the number of unique synthetic internal standards is equal to 4\hat{ }n,

According to the above method, n is the number of N positions, and the number of N needed for the sDNA sample is calculated by log (X)/log(4),

According to the above method, X is the genome equivalence for the sDNA sample; According to the above method, the N positions are minimized to the degree possible based upon the size of the genome of the sDNA sample.

According to the above method, the number of N position is more than expected number of sDNA templates.

According to the above method, a single unique base change may be substituted in adjacent to the N region of the IS to facilitate bioinformatics identification of IS sequences during sequencing and analysis.

According to the above method, the NGS library preparation is an amplicon based or a hybrid capture based NGS library preparation procedure.

According to the above method, a more accurate limit of detection for a gene target in the gDNA sample can be made by identifying the number of templates captured in NGS library.

According to the above method, the complexity yield which is significantly lower than a previously established normal complexity yield indicates stochastics errors.

A method of determining the deduplication efficiency, comprising obtaining an amount of sample DNA (sDNA) comprising one or more endogenous target genes; preparing a set of synthetic internal standards for at least one of said target genes, wherein the internal standards are the sequence of the target gene within which a substitution of a number (n) of adjacent bases with all 4 nucleic acid bases (N) is made; comingling a known copy number of the internal standards with the sDNA sample to create a combined sample; sequencing the combined sample before a deduplication process; analyzing the sequencing data to measure the number of unique reads corresponding to the internal standards (pre-deduplication unique IS); deduplicate the combined sample; sequencing the combined sample after the deduplication process; analyzing the sequencing data to measure the number of unique reads corresponding to the internal standards (post-deduplication unique IS); determine the deduplication efficiency by comparing the pre-deduplication unique IS and the post-deduplication unique IS.

### BRIEF DESCRIPTION OF THE DRAWINGS

FIG. 1 depicts the results of an example complexity capture QC of 2000 genome equivalence of SARS-CoV-2 IS added to serial dilutions of TWIST COVID-19 Reference RNA and sequenced using ARTIC v3 protocol.

FIG. 2 depicts the use of the NT:IS ratio and complexity control to estimate the sequence representation of each target region in the original sample. The upper sample (circles) depict 98 tiled amplicon (x-axis) yields (y-axis) adjusted for complexity capture for three samples (lines).

FIG. 3 depicts the use of NT amplicon base counts for the same samples shown in FIG. 2. This figure demonstrates that, without the claimed methods, it is unclear that exon 16 and 17, with >200,000 base counts, for the lower sample had insufficient complexity capture as compared to FIG. 2

FIG. 4 depicts the profile complexity loss profile arising from unique molecular tag error correction due to deduplication as a measure of the unique reads count pre- and post-tag deduplication.

### DETAILED DESCRIPTION OF THE INVENTION

The following is a detailed description provided to aid those skilled in the art in practicing the present disclosure. Those of ordinary skill in the art may make modifications and variations in the embodiments described herein without departing from the spirit or scope of the present disclosure. All publications, patent applications, patents, figures and other references mentioned herein are expressly incorporated by reference in their entirety.

Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either both of those included limits are also included in the disclosure.

All numerical values within the detailed description and the claims herein are modified by “about” or “approximately” the indicated value, and take into account experimental error and variations that would be expected by a person having ordinary skill in the art.

The following terms are used to describe the present disclosure. Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, “nucleic acid” can refer to a polymeric form of nucleotides and/or nucleotide-like molecules of any length. In certain embodiments, the nucleic acid can serve as a template for synthesis of a complementary nucleic acid, e.g., by base-complementary incorporation of nucleotide units. For example, a nucleic acid can comprise naturally occurring DNA, e.g., genomic DNA; RNA, e.g., mRNA, and/or can comprise a synthetic molecule, including but not limited to cDNA and recombinant molecules generated in any manner. The terms “polynucleotide,” “polynucleotide molecule,” “nucleic acid molecule,” “polynucleotide sequence” and “nucleic acid sequence,” can be used interchangeably with “nucleic acid” herein. In some specific embodiments, the nucleic acid to be measured may comprise a sequence corresponding to a specific gene.

The term “native template” as used herein can refer to nucleic acid obtained directly or indirectly from a specimen that can serve as a template for amplification. For example, it may refer to cDNA molecules, corresponding to a gene whose expression is to be measured, where the cDNA is amplified and quantified.

The preparation of a sequencing library involves some combination, or all, of the following steps: 1) nucleic acid fragmentation; 2) in vivo cloning, which serves to attach flanking nucleic acid adaptor sequences; 3) in vitro adaptor ligation; 4) PCR based adaptor addition; and, 5) unimolecular inversion probe type technology with, or without, polymerase fill-in, and ligation of probe to capture the sequence by circularization, with adaptor contained within the probe sequence.

A given nucleic acid target (NT) from within the sample to be sequenced is selected. Each nucleic acid target is similar to a respective internal standard, with the exception of one or more changes to the nucleic acid sequence. These differences between native target and internal standard are identifiable with sequencing, and can include deletions, additions, or alteration to the ordering or composition of nucleotides used.

In order to calculate the complexity yield, the internal standards used in this method made where several adjacent positions are synthesized with all four nucleotides (N). The resulting number of unique control sequences (CC-IS) would be equal to 4\hat{ }n, where n is the number of N positions. In an embodiment too many N positions can lead to the control not behaving biochemically equivalent to the sample, so it is desirable to minimize the number of N positions. The number of n required to accurately measure complexity depends on the input level of sample. For example, 30,000 genome equivalence sample input would require 8 N positions (CIELING (log(30000)/log(4))). A single unique base change may be added in/adjacent to the N region to facilitate bioinformatic identification of control.

In certain embodiments, the complexity yield can be calculated using this method where several non-adjacent positions are synthesized with all four nucleotides (N).

The number of times a given nucleotide in a sample is sequenced is referred to as coverage. Coverage is variable within a sample, and deeper coverage of a given nucleotide provides a more reliable determination of the identity of the nucleotide at a given position. The terms “depth of coverage”, “coverage depth” or “target coverage”, as used herein, refer to the number of sequenced DNA fragments (e.g., reads) that map to a given genomic target. The deeper the coverage of a target region (e.g., the more times the region is sequenced), the greater the reliability and sensitivity of the sequencing assay. In general, low frequency, or rare, sequence variations require greater depth of coverage in order to be detected in a sample.

Complexity yield, as used herein, is a measure of the capture efficiency of the NGS library during its preparation. Known methods for controlling NGS library preparation such as adding unique molecular identifiers are useful for indicating unique input templates, but do not estimate how well the original sample complexity was captured in the library. In contrast, calculating the complexity yield estimates how many target sequences made it into the NGS library following its preparation. The Complexity yield is calculated by taking the total unique complexity reads of the N region (CC-IS) divided by the known number of copies of the control spiked into the sample.

$$Complexity Yield = \frac{Unique CC - IS Count}{Control Input Copies} \qquad Eq . 1$$

The complexity yield can be combined with the control to provide a measure of input abundance. Input abundance is the number of sample templates captured in the NGS library, calculated by dividing the product of the total number of native target sequences read (NT) and the known number of copies of the control spiked into the sample by the total number of control sequences read (IS).

$$Input Abundance = \frac{NT \times Control Input Copies}{IS count} \qquad Eq . 2$$

The sample target template number in the library is a calculated measure of the amount of sequences in the NGS library preparation.

Sample Template Captured by Library=Input Abundance×Complexity Yield Eq. 3

The number of templates captured for a given library is a means of informing the limit of detection (LOD) or variant allele frequency (VAF) sensitivity for a given sample target.

The term “variant calling”, as used herein, refers to the process of determining if a sequence variation is a true variant derived from the original sample, and thus used in the analysis, or the result of a processing error and thrown out. For example, some variant calling algorithms require at least 3 unique reads to call a variant positive.

### Example 1

The complexity yield of a prepared NGS library is calculated according to Equation 1. 30,000 copies of controls were spiked into the NGS library during preparation. Sequence analysis results in 2,300 unique control reads. According to Equation 1, the complexity yield of this NGS library is 2,300÷30,000=0.076.

Sequence analysis of the NGS library results in 6,666 native reads and 40,000 total control reads. The input abundance is calculated according to Equation 2, (6,666÷(40,000×30,000))=5,000 copies of target sample. The sample template is calculated according to Equation 3, (0.076×5,000)=383 sample templates were captured in the NGS library.

### Example 2

The complexity yield of a prepared NGS library is calculated for a single target gene (T1). The complexity yield is then applied across three other targets within the NGS library (T2-T4) to provide for an adjusted NT input level (row 10) for each target.

TABLE 1

| | | T1 | T2 | T3 | T4 |
|---|---|---|---|---|---|
| 1 | Total IS Reads | 17599 | 26201 | 23388 | 19030 |
| 2 | Total NT Reads | 16480 | 36867 | 29215 | 19493 |
| 3 | Unique Control | 8000 | | | |
| | Sequences for | | | | |
| | T1-CC-IS | | | | |
| 4 | correction factor | 1.00 | 0.67 | 0.75 | 0.92 |
| 5 | Input Abundance—IS | 8000 | 5333 | 6000 | 7333 |
| 6 | Input Abundance—NT | 7491 | 7504 | 7495 | 7512 |
| 7 | Number of Internal | 30000 | | | |
| | Standards spiked-in | | | | |
| 8 | Library efficiency | 27% | | | |
| 9 | IS input level | 30000 | 20000 | 22500 | 27500 |
| 10 | NT input level | 28093 | 28142 | 28106 | 28169 |

The values in Rows 1 and 2 are experimentally determined. Row 6 is the Input Abundance as calculated in Eq. 2. The IS input level (row 9) is known for each target, and the correction factor (row 4) is a ratio of each target's IS input level divided by the IS input level of T1, and the Input Abundance—IS (row 5) is the product of the correction factor for each target multiplied by the CC-IS for T1.

The unique sequence count can also be used to evaluate the efficiency of the deduplication efficiency. By comparing the unique sequence counts before and after the deduplication process, accuracy of deduplication can be tested. For example, a particular NNNN sequence (AGCC) has 25 reads before the deduplication process, and 3 reads after the deduplication process. Therefore, it is clear that the deduplication process did not accurately reduce the reads to 1 read. Alternatively, one can detect 2000 different unique sequence counts before the deduplication process, and 1100 reads after the deduplication process. This reflects that the deduplication resulted in a loss in complexity yields.

Monte carlo simulations is used to estimate the expected frequency of an NNNN population for a given total coverage. By comparing the expected frequency with the NNNN complexity before the deduplication and the duplication rates measured by NGS, it indicates how well the controls are represented in the raw library reads. The difference from estimated frequency can indicate defects in oligo synthesis of complexity controls.

### Example 3—Complexity Control Quality Assurance of Sample Preparation

SARS-CoV-2 SNAQ-SEQ IS and CC were created to be compatible with the ARTIC V3 library preparation (Wellcome Sanger Institute—COVID-19 ARTIC v3 Illumina library construction and sequencing protocol V.4 accessible at the website:protocols.io/view/covid-19-artic-v3-illumina-library-construction-an-bgxjjxkn). The IS would allow quantification of each viral genome positions corresponding to each amplicon location and the CC would provide monitoring of complexity capture. SARS-CoV-2 SNAQ-SEQ IS corresponded to the entire SARS-CoV-2 sequence, with 17 overlapping RNA contigs that matched the Wuhan SARS-CoV-2 sequence (MN908947.3) except for complementary base changes every 50 positions. Base changes falling under ARTIC v3 primer binding sites were repositioned outside primer binding sites, while ensuring at least 2 base changes per amplicon. An RNA molecule corresponding to positions 5100-6104 contained two CC placed within the regions corresponding to Arctic_V3_18 and Arctic_V3_19 PCR amplicons. Each CC had 8 degenerate bases adjacent to a complementary base change.

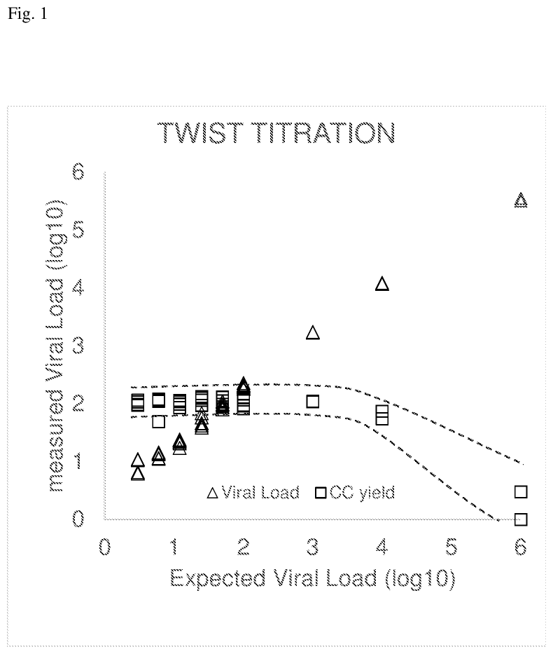

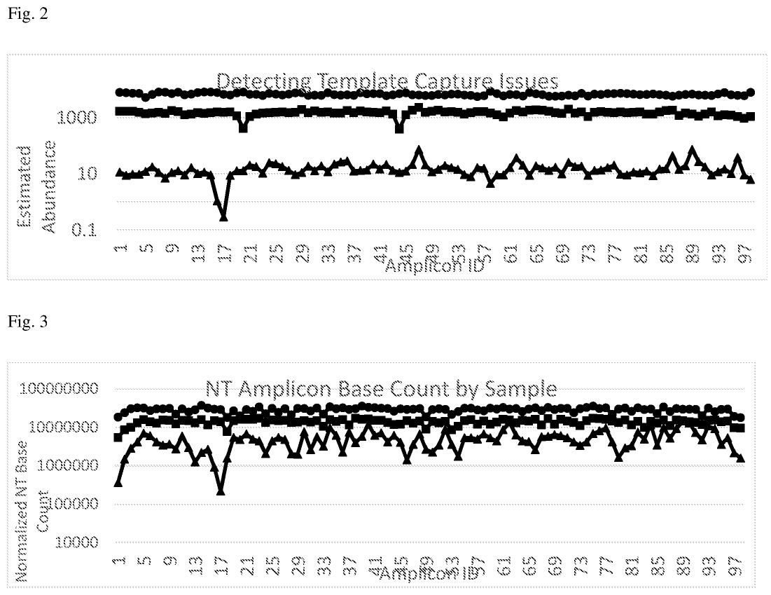

2000 SARS-CoV-2 SNAQ-SEQ IS and CC were added to different levels of SARS-CoV-2 TWIST reference RNA (FIG. 1 x-axis) prior to ARTIC V3 library preparation and sequenced on an Illumina NextSeq instrument. FASTQ sequence were aligned using BWA mem and a Wuhan SARS-CoV-2 reference sequence (MN908947.3) appended with the SNAQ-SEQ IS and CC contigs. An awk script was used to count IS and NT amplicons, extract the CC degenerate sequences and their flanking bases (positions 5531-5540, and 5753-5807) and count unique complexity sequences (FIG. 1, squares). Only CC sequences with the expected size (10 bases) and expected outside bases were counted. For each viral sample, the average ratio of NT:IS amplicon count was used to calculate viral load (FIG. 1, triangles). For three representative samples, the NT:IS ratio was used to estimate the genomic copies for positions corresponding to each amplicon (FIG. 2) and the estimated their genomic representation by adjusting for CC yield (amplicon_abundance*CC_yield, FIG. 2, y-axis).

The fraction of CC template were calculated as a ratio of unique CC reads divided by CC input level (e.g., 400/2000), or around 20% for this library preparation. The CC were designed to biochemically mimic the NT, thus it is expected the overall capture of input NT genomes was 20%.

An example of complexity capture QC acceptance criterion may be calculated from reference samples. For example, 99.5% confidence interval derived from average and standard deviation of different viral load levels may be established during assay development. These confidence intervals are represented as dashed lines in the plot (FIG. 1), samples with CC results falling outside the parallel dashed lines are suspected as having yield issues. The inventors believe, without being bound by theory, that it is possible to combine the CC yield for the entire run (e.g, by binning half log viral load CC results) and compare the results with the pre-established CC mean to provide a highly sensitive way to detect method drift before it manifests as sample failure.

### Example 3B—Improve Variant Calling by Eliminating Extra Reads

To improve the statistics of variant detection, it is desirable to down sample reads to 1 read per input template. For sequence methods unable to collapse replicate reads to a single read per captured template, some level of down sampling is beneficial to minimize the impact of replicate reads on variant calling statistics. Crudely, down sampling read depth to 6-fold higher than genomic input attempts to balance loss of complexity due to stochastic sampling with over sampling of reads. For the samples depicted in the plot, the viral load may be used to indicate the level to down sample sequence reads. For example, a sample with an average read depth of 40,000 and a 2000 viral genomes added to library preparation, should be down sampled to 12,000 read depth (2000 genomes×6-fold coverage). The CC offers an additional improvement to down sampling by also factoring the complexity capture efficiency into the down sampling calculation. The previous example should be further down sampled 20% (2400 coverage) to reflect the samples sequence complexity more accurately.

### Example 4—Region Specific Monitoring of Complexity Capture

The NT:IS ratio may be used to estimate the abundance of each target region in the original sample. Combining abundance estimate with complexity capture generates a clearer picture of sample genomic representation. The abundance in the original sample may be estimated from the NT:IS ratio. However, this is NOT the amount of template detected in the sequencing reads because the efficiency of complexity capture needs to be factored in.

For example, in FIG. 2, 100 templates in original sample were reduced to 10 templates based on the 10% complexity capture yield. The upper sample (circles) depict 98 tiled amplicon (x-axis) yields (y-axis) adjusted for complexity capture for three samples (lines), the top sample did not display any abnormal amplicon capture. The middle sample (squares) had two abnormal amplicon yields arising from SNV in primer binding site, resulting in 3-fold lower coverage (regions 20 & 44). The lower sample (triangles), had 100 viral genomes in sample, but had low template captured as sequence and experienced high stochastic variation in coverage. Viral load without the CC correction would overestimate possible coverage, leading to poor assumptions about LOD. For this sample, two low amplicon yields arising from SNV in primer binding site resulted in very low coverage of two regions (15 & 16), but unlike the previous sample, the reduced yield will significantly impact variant detection for these target regions, with an estimated complexity of a single genome.

The lower plot (FIG. 3) depicts what is currently available for coverage QC, the base counts per amplicon for the same three samples (same relative order). From this figure, it is unclear that exon 16 and 17, with >200,000 base counts, for the lower sample had insufficient complexity capture.

### Example 5—Profile Complexity Lost due to Unique Molecular Tag Deduplication

A common practice is to attach unique molecular tags to ends of the original sample templates prior to any template replication event. Library preparations then replicate the original templates and its associated unique tag. This approach is used for sequencing error correction though building a consensus sequence from replicate reads. The complexity control may be used to determine the loss of complexity capture due to the deduplication event. Crudely, the unique reads count pre and post tag deduplication will indicate how much complexity was lost.

Two dsDNA complexity controls were synthesized with sequence corresponding to two amplicons of a custom Ampliseq-HD NGS library preparation, one control for an amplicon in PCR pool 1, the second control in PCR pool 2. Each control had 7 contiguous degenerate bases adjacent to a single complementary base change, roughly centered in middle of the amplified sequence and a second complementary base change 5 bases into one end of the amplified sequence. The two complementary base changes were used to enable identification of the control during alignment and the 7 degenerate bases would provide 16384 different control sequences. A stock of 4000 CC per μl was created. 1 μl of controls were added to purified DNA sample (approximately 8000 genomes) prior to library preparation and deep sequenced. A proprietary bioinformatic pipeline was used to extract the complexity control reads pre and post UMI deduplication. The loss of complexity due to UMI deduplication was binned as a function of their pre deduplicated replicate level.

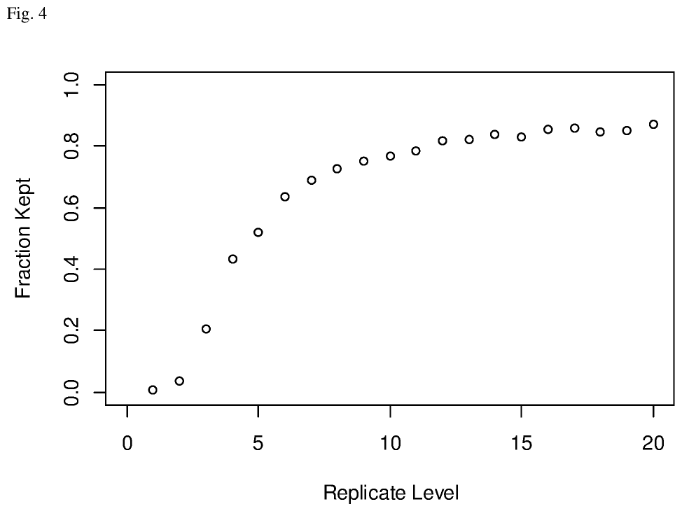

The plot (FIG. 4) provides more details complexity loss arising from deduplication. The more the original template was replicated (x-axis) the better chance the original template was present post deduplication (y-axis). In this example, an original template had a >50% chance of being present in the deduplicated reads when it had 5 or more reads. >11 replicate reads were required to have 80% of being represented in the deduplicated reads. For these samples, >38% and 58% templates were replicated 5 or 11, respectively. The impact of deduplication on complexity capture may be used to either optimize assay, or monitor deduplication efficiency to detect method drift.

What is claimed:

1. A method of determining the complexity yield of a prepared next generation sequencing (NGS) library, comprising
obtaining an amount of sample DNA (sDNA) comprising one or more endogenous target genes;
preparing a set of synthetic internal standards for at least one of said target genes, wherein the internal standards are the sequence of the target gene within which a substitution of a number (n) of adjacent bases with all 4 nucleic acid bases (N) is made;
comingling a known copy number of the internal standards with the sDNA sample to create a combined sample;
preparing a NGS library from the combined sample for sequencing;
sequencing the combined sample; and
analyzing the sequencing data to measure the number of unique reads corresponding to the internal standards (unique IS), and calculating a complexity yield between the unique IS and the known copy number of internal standards in the combined sample.

2. The method of claim 1, wherein the complexity yield indicates the quality of the prepared NGS library;
wherein a reduce of the complexity yield from a previously established normal complicity yield indicates a poorer quality of the prepared NGS library.

3. The method of claim 1, wherein a number of sDNA target templates in the NGS library prepared from the combined sample is calculated by multiplying the ratio of unique IS to a total number of control reads (IS depth) with the total number of native reads (NT depth),
wherein the number of sDNA templates in the NGS library is used to calibrate the amount of sDNA needed in the preparation of a second NGS library to provide independent biochemical depth analysis for the one or more endogenous target genes in the sDNA than in the original NGS library.

4. The method of claim 1, wherein the amount of sDNA in the second NGS library is adjusted to provide an adequate number of unique reads of one or more endogenous target genes to provide variant allele frequency sensitivity.

5. The method of claim 1, wherein the number of unique synthetic internal standards is equal to 4\hat{ }n,
wherein n is the number of N positions, and the number of N needed for the sDNA sample is calculated by log (X)/log(4),
wherein X is the genome equivalence for the sDNA sample;
wherein the N positions are minimized to the degree possible based upon the size of the genome of the sDNA sample.

6. The method of claim 5, wherein the number of N position is more than expected number of sDNA templates.

7. The method of claim 1, wherein a single unique base change may be substituted in adjacent to the N region of the IS to facilitate bioinformatics identification of IS sequences during sequencing and analysis.

8. The method of claim 1, wherein the NGS library preparation is an amplicon based or a hybrid capture based NGS library preparation procedure.

9. The method of claim 1, wherein a more accurate limit of detection for a gene target in the gDNA sample can be made by identifying the number of templates captured in NGS library.

10. The method of claim 1, wherein the complexity yield which is significantly lower than a previously established normal complexity yield indicates stochastics errors.

11. A method of determining the deduplication efficiency, comprising
obtaining an amount of sample DNA (sDNA) comprising one or more endogenous target genes;
preparing a set of synthetic internal standards for at least one of said target genes, wherein the internal standards are the sequence of the target gene within which a substitution of a number (n) of adjacent bases with all 4 nucleic acid bases (N) is made;
comingling a known copy number of the internal standards with the sDNA sample to create a combined sample;
sequencing the combined sample;
analyzing the sequencing data before a deduplication process to measure the number of unique reads corresponding to the internal standards (pre-deduplication unique IS);
analyzing the sequencing data post deduplication to measure the number of unique reads corresponding to the internal standards (post-deduplication unique IS);
comparing replicate counts of each unique sequence pre and post deduplication to evaluate the efficiency and errors of deduplication step.

12. The method of claim 1, wherein the substitution of a number (n) of adjacent bases with all 4 nucleic acid bases (N) of the internal standards are different from the sequence of the target gene.

13. The method of claim 12, wherein the number of N needed for the sDNA sample is less than log (X)/log(4), wherein X is the genome equivalence for the sDNA sample.
